(19) United States
(12) Patent Application Publication
(10) Pub. No.: US 2018/0104309 A1
(43) Pub. Date: Apr. 19, 2018

(54) COMPOSITIONS AND METHODS FOR SPINAL CORD REGENERATION

(71) Applicant: DUKE UNIVERSITY, Durham, NC (US)

(72) Inventors: Kenneth D. POSS, Chapel Hill, NC (US); Mayssa H. MOKALLED, Durham, NC (US)

(21) Appl. No.: 15/573,415

(22) PCT Filed: May 11, 2016
(86) PCT No.: PCT/US2016/031860

§ 371 Date: Nov. 10, 2017

Related U.S. Application Data
(60) Provisional application No. 62/159,413, filed on May 11, 2015.

(51) Int. Cl. A61K 38/18; C07K 14/475

(52) U.S. Cl. CPC A61K 38/18; C07K 14/475

(57) ABSTRACT

The invention provides pharmaceutical compositions and methods of use thereof for ameliorating injuries and conditions of the central nervous system. More specifically, the invention provides pharmaceutical compositions that when administered promote spinal cord regeneration.

### BACKGROUND OF THE INVENTION

### Field of the Invention

The present disclosure relates generally to field of neurobiology. Specifically, the present disclosure relates to compositions and methods of overcoming the complications of mammalian spinal cord injury. More particularly, the disclosure provides compositions and methods for promoting efficient axon growth, adult neurogenesis, and preventing glial scarring formation for treating neuronal injury, and more particularly spinal cord injury.

### Description of Related Art

Whereas extensive research is focused on overcoming the complications of mammalian spinal cord (SC) injury, the mechanisms that direct natural SC repair remain comparatively unexplored. Adult zebrafish are capable of spontaneous recovery after SC injury (Reimer et al. (2008) J Neurosci 28, 8510-8516; T. Becker et al. (1997) J Comp Neurol 377, 577-595; Kizil et al. (2012) Dev Cell 23, 1230-1237; Hui, et al. (2010) Dev Dyn 239, 2962-2979.) At 1-week post injury (wpi), fish are totally paralyzed caudal to the injury site and spend most of their time at the bottom of the tank. They activate a regenerative program that enables them to regain sensory and motor function. And by 8 wpi, they are swimming around normally in the tank.

In contrast, the mammalian SC undergoes a series of primary and secondary damages after SC injury. These damages resolve themselves by forming a scar that is inhibitory for axon growth or neurogenesis. Efficient axon growth, adult neurogenesis, and absence of glial scarring distinguish the injury response of the zebrafish SC from mammalian SCs. (Reimer et al. (2008) J Neurosci 28, 8510-8516; T. Becker et al. (1997) J Comp Neurol 377, 577-595; Hui, et al. (2010) Dev Dyn 239, 2962-2979.)

Following complete SC transection in zebrafish, inflammatory responses are thought to trigger the initial steps of regeneration (Kyritsis et al. (2012) Science 338, 1353-1356), which include ependymal cell proliferation at the lesion site (see FIG. 1A). Next, a bridge of glial cells connects the two severed SC portions. (Goldshmit et al. (2012) J Neurosci 32, 7477-7492.) Bridging is a striking, pro-regenerative glial response that is thought to provide a natural scaffold for axonal growth. (Id.) At this step, glial cells connect the transected SC and form a bridge along which axons will grow. Goldshmit et. al. (2012) were the first to describe glial bridging, showing that the bridge is formed by GFAP-positive cells. However, GFAP is a global marker of glia and radial glia. With the lack of a specific way to mark bridging glia, the origin and type of glia that construct the bridge remained unknown.

Injury to the mammalian central nervous system (CNS) results in irreversible impairment of sensory and motor functions. Following spinal cord (SC) injury, adult zebrafish (1) initiate a glial bridge that connects the transected parts of the cord; (2) regenerate axon tracts along bridging glia; and (3) generate new neurons. The studies described herein identify proteins that facilitate and promote spinal cord injury repair.

### SUMMARY OF THE INVENTION

It is against the above background that the present invention provides certain advantages and advancements over the prior art.

Although embodiments of the invention disclosed herein are not limited to specific advantages or functionalities, in embodiments the invention provides a pharmaceutical composition comprising proteins or peptides useful for regenerating neuronal tissue, and more specifically, spinal cord tissue.

In some aspects, the pharmaceutical composition disclosed herein comprises: a protein or peptide wherein the protein or peptide is capable of initiating glial bridge formation, regenerating axon tracts along bridging glia, and/or generating neuron formation.

In some aspects of the pharmaceutical composition disclosed herein, the protein or peptide capable of initiating glial bridge formation, regenerating axon tracts along bridging glia, and or generating neuron formation comprises a protein encoded by vertebrate or mammalian fn1b, ctgf, mstnb, stc1l, ccl44, or uts2b genes.

The invention provides a pharmaceutical composition comprising connective tissue growth factor (“CTGF”) proteins or peptides useful for regenerating neuronal tissue, and more specifically, spinal cord tissue.

In some aspects of the pharmaceutical composition disclosed herein comprises a pharmaceutically acceptable carrier suitable for intravenous, intramuscular, oral, intraperitoneal, intradermal, transdermal, topical, or subcutaneous administration.

The disclosure also provides a method of regenerating spinal cord tissue in a subject in need thereof, the method comprising administering a therapeutically effective amount of CTGF peptide.

The present disclosure also provides a method of ameliorating spinal cord injury in a subject in need thereof, the method comprising administering a therapeutic effective amount of CTFG peptide or protein.

The present disclosure also provides a method of initiating glial bridge formation, regenerating axon tracts along bridging glia, and/or generating neuron formation in a tissue of a subject in need thereof by administering CTFG peptide.

In some aspects of the method of ameliorating neuronal injury, the injured tissue is spinal cord tissue.

The present disclosure also provides a method of neuronal tissue, and particularly regenerating spinal cord tissue in a subject in need thereof, the method comprising administering a therapeutic effective amount of a protein or peptide initiating glial bridge formation, regenerating axon tracts along bridging glia, and/or generating neuron formation.

In some aspects of the method of regenerating spinal cord tissue, the protein comprises a protein encoded by the fn1b, ctgf, mstnb, stc1l, ccl44, or uts2b gene.

In some aspects, the disclosure relates to ameliorating and/or repairing spinal cord injury in a mammal.

In some aspects, the disclosure relates to ameliorating and/or repairing spinal cord injury in a human.

The present disclosure also provides a kit useful for the treatment of spinal cord injury in a subject, the kit comprising a therapeutically effective amount of a pharmaceutical composition for the regeneration of spinal cord tissue and instructions for use.

Specific preferred embodiments of the present invention will become evident from the following more detailed description of certain preferred embodiments and the claims.

### BRIEF DESCRIPTION OF THE DRAWINGS

The following detailed description can be best understood when read in conjunction with the following drawings in which:

FIG. 1A shows an overview of the multi-step process of SC regeneration. FIG. 1B shows a screen for secreted factors increased during SC regeneration; RNA-sequencing of injured SC tissues at 2 weeks post-injury (wpi) identified 2090 genes with increased expression. Expression and regulation cutoffs of 20 fpkm and 4-fold filtered 76 genes with the requisite expression and fold-change. Gene Ontology classification revealed seven out of the 76 increased genes that encode secreted, extracellular proteins. FIG. 1C shows expression of those genes (7) increased around the lesioned area of the injured SC.

FIG. 2 shows molecular genetic (A-C) and histologic (D-F) analyses of the spatiotemporal pattern of ctgfa expression in the absence (Control) or at 1 or 2 weeks post-injury (wpi). Dotted lines delineate the central canals. Scale bars, 50 μm. ctgfa:EGFP expression is indicated by the light-colored fluorescence signal localized to the ventral region of the central canal (indicated by arrows).

FIG. 3 shows in situ hybridization analysis of ctgfa expression on longitudinal sections of the rostrocaudal SC axis in the absence of injury (control, A), or 1 week (B), 2 weeks (C), or 3 weeks (D) post-injury. Dotted lines delineate the periphery of the spinal cord. Scale bars, 50 μm. At 2 wpi, ctgfa transcripts localize to ventral ependymal cells (arrowheads) and to the cellular bridge connecting the transected SC (arrows).

FIG. 4 shows the results of expression analyses using a ctgfa reporter construct (ctgfa:EGFP) in cells co-stained with GFAP. ctgfa:EGFP reporter expression co-localized with GFAP during early bridging events at 5 days post-injury (dpi) (A, bright region in box, blown up and presented in B), and after bridge formation at 2 wpi (E). Dotted lines delineate SC edges, and arrows point to the sites of bridging. A high magnification, longitudinal view of the boxed area from panels A and B shows the elongated morphology of the first detectable CTGF⁺GFAP⁺ bridging glia (C and D). Arrows indicate presence of the EGFP that localizes to the cellular bridge connecting the transected SC.

FIG. 5A shows a schematic of the wild-type Ctgfa protein and the ctfga genomic locus, and indicates the location of, and sequence change in, the ctfga^{bns50} mutant locus. Shown are the domain structure of Ctgfa protein including a protease domain (arrowhead), and the genomic organization of ctgfa wild-type (ctgfa⁺) and mutant (ctgfa^{bns50}) loci. FIG. 5B shows ctgfa mutant fish have normal baseline swim capacity (no change relative to wild-type or heterozygous mutant). Adult uninjured wild-type (ctgfa+/+), heterozygous (ctfga^{bns50/}+), and mutant (ctfga^{bns50/bns50}) animals possess comparable swim capacities. For statistical analyses, (ns) indicates non-significant P-value >0.05.

FIG. 6, A-C show acetyl-α-tubulin immunohistochemistry on wild-type (ctgfa^{+/+}), ctgfa heterozygous (ctgfa^{bns50/+}) and mutant (ctgfa^{bns50/bns50}) clutchmate fish. At 4 wpi, ctgfa mutant fish failed to regenerate compared to control fish (see bright acetyl-α-tubulin staining in punctate/fibrous pattern within the briding structures). For histology, dotted lines delineate SC edges, and arrows point to the sites of bridging. Scale bars, 100 μm. FIG. 6D shows impaired motor function recovery following CTGF loss-of-function. Swim assays determined the exhaustion points (minutes) for 9 wild-type, 11 heterozygous and 10 mutant animals under increasing water current at 4 wpi. For statistical analyses, (*) and (**) represent P-values of <0.05 and <0.01, respectively.

FIG. 7 shows generation of ctgfa overexpressing fish. (A) schematic of ctgfa and EGFP cassettes subcloned downstream of the hsp70 heat shock-inducible promoter to generate hsp70:ctgfa transgenic animals. (B, C) EGFP expression in hsp70:ctgfa SCs prior to (B) and 2 days post-heat shock treatment (C). Inducible expression of the ctgfa transgene indicated by the bright (EGFP) signal in heat shock treated SC. Scale bars, 50 μm.

FIGS. 8, A and B show ctfga enhanced SC regeneration. Acetyl-α-tubulin immunohistochemistry on ctgfa-overexpressing (B) and transgene-negative control animals (A). At 2 wpi, hsp70:ctgfa^{Line1} fish show increased regeneration compared to control fish (see bright acetyl-α-tubulin staining in punctate/fibrous pattern within the bridging structures). For histology, dotted lines delineate SC edges, and arrows point to the sites of bridging; scale bars, 100 μm. FIG. 8C shows enhanced motor function recovery after ctgfa overexpression. Swim assays determined the exhaustion points (minutes) for 12 control and 12 ctgfa overexpressing fish under increasing water current at 2 wpi. For statistical analyses, (*) represents a P-value of <0.05.

FIG. 9 shows enhanced bridging and regeneration following CTGF overexpression. (A, B, B′) lmmunohistochemistry for acetyl-α-tubulin and GFAP was performed on longitudinal sections of SC from ctgfa-overexpressing and transgene-negative control animals. At 2 wpi, hsp70:ctgfa (line 2) animals show increased regeneration compared to control fish (bright acetyl-α-tubulin staining) (dotted lines delineate SC edges, and arrows point to the sites of bridging). Scale bars, 100 μm. (C) Quantification of glial bridging at 2 wpi: percent bridging was calculated for 3 longitudinal SC sections per fish, and averaged for a total of 5 control, 6 hsp70:ctgfa (line 1), and 8 hsp70:ctgfa (line 2) animals. For statistical analyses, (*) represents a P-value of <0.05, and ns represents P-value >0.05.

FIG. 10 shows that CTGF protein is highly conserved in vertebrates. (A) Domain structure of the CTGF protein. In addition to a signal peptide (SP), CTGF harbors four protein interaction motifs: an Insulin-like growth factor-binding domain (IGFB), a Von Willebrand factor type C repeat (VWC), a Thrombospondin type 1 repeat (TSP1), and a Cysteine knot domain (Cys knot). In addition, CTGF possesses a protease domain marked by an arrowhead. (B) Zebrafish Ctgfa and human CTGF are 77% identical and 85% similar. Identity and similarity increase to 81% and 87% within domains, respectively. (C) Amino acid alignment for human CTGF, mouse CTGF, and the zebrafish orthologues Ctgfa and Ctgfb. Identical and similar amino acids are highlighted in black and grey, respectively.

FIG. 11 shows human recombinant CTGF (HR-CTGF) treatment promotes SC regeneration. Acetyl-α-tubulin immunohistochemistry shows enhanced SC repair at 2 wpi in HR-CTGF-treated animals (B) compared to vehicle controls (A). For histology, dotted lines delineate SC edges, and arrows point to the sites of bridging. Scale bars, 100 μm. FIG. 11C shows enhanced motor function recovery after HR-CTGF treatment. Swim assays determined the exhaustion points (minutes) for 9 control, 9 HR-CTGF-treated and 6 uninjured fish under increasing water current at 2 wpi. For statistical analyses, (**) represents P-values of <0.01.

FIG. 12 shows Immunohistochemistry for GFAP on SC longitudinal sections from wild-type (ctgfa^{+/+}, A, D), ctgfa heterozygous (ctgfa^{bns50/+}, B, E) and mutant (ctgfa^{bns50/bns50}, C, F) clutchmate fish. GFAP staining comprises lighter punctate/fibrous pattern, and is a marker of SC regeneration (Goldshmit et al. (2012) J Neurosci 32, 7477-7492). At 4 wpi, ctgfa mutants failed to regenerate compared to control fish (A-C). For histology, dotted lines delineate SC edges, and arrows point to the sites of bridging. Scale bars, 100 μm. (G) Quantification of glial bridging in ctgfa mutant fish at 2 wpi. Percent bridging was calculated for 3 longitudinal SC sections per fish, and averaged for a total of 6 wild-type, 4 heterozygous, and 4 mutant animals. For statistical analyses, (**) represents P-values of <0.01, while (ns) indicates non-significant P-value >0.05.

FIG. 13 shows GFAP immunohistochemistry indicating increased GFAP expression and enhanced glial bridging in HR-CTGF treated animals (B, D, F) at 1 and 2 wpi compared to vehicle controls (A, C, E). Dotted lines, arrows, and scale are the same as in FIG. 12.

FIG. 14 shows quantification of glial bridging at 1 wpi. Percent bridging was calculated for 3 longitudinal SC sections per fish, and averaged for 8 control and 8 treated animals. For statistical analyses, (*) represents a P-value of <0.05.

FIG. 15 shows HR-CTGF treatment induces neurite outgrowth in vitro.

FIG. 16 shows expression of ctgf after mouse spinal cord hemisection injury. Ctgf expression indicated by the dark staining pattern in the lower panels.

### DETAILED DESCRIPTION OF THE INVENTION

All publications, patents and patent applications cited herein are hereby expressly incorporated by reference for all purposes. Unless otherwise defined, all technical terms used herein have the same meaning as commonly understood by one of ordinary skill in the art to which this disclosure belongs.

### Definitions

Before describing the disclosed methods and compositions in detail, a number of terms will be defined. As used herein, the singular forms “a”, “an”, and “the” include plural referents unless the context clearly dictates otherwise. In other words, the articles “a” and “an” are used herein to refer to one, or to more than one (i.e., at least one), of the grammatical object of the article. By way of example, “an element” means at least one element and can include more than one element.

It is noted that terms like “preferably,” “commonly,” and “typically” are not utilized herein to limit the scope of the claimed invention or to imply that certain features are critical, essential, or even important to the structure or function of the claimed invention. Rather, these terms are merely intended to highlight alternative or additional features that can or cannot be utilized in a particular embodiment of this invention.

For the purposes of describing and defining this invention it is noted that the term “substantially” is utilized herein to represent the inherent degree of uncertainty that can be attributed to any quantitative comparison, value, measurement, or other representation. The term “substantially” is also utilized herein to represent the degree by which a quantitative representation can vary from a stated reference without resulting in a change in the basic function of the subject matter at issue.

As used herein, the term “subject” and “patient” are used interchangeably herein and refer to both human and nonhuman animals. The term “nonhuman animals” of the disclosure includes all vertebrates, e.g., mammals and non-mammals, such as nonhuman primates, sheep, dog, cat, horse, cow, chickens, amphibians, reptiles, and the like. Preferably, the subject is a human patient.

As used herein, the terms “disorder of the nervous system” and “injury to the nervous system” can be used interchangeably in reference to a physical condition relating to the brain and/or nervous system including the spinal cord. Such conditions include, but are not limited to, stroke, trauma, neurodegenerative disease, and physical injury.

As used herein, the term “spinal cord” refers to a cylindrical, bilaterally symmetrical, segmented nerve cord that begins at the base of the brainstem and ends in a tapered structure called the conus medullaris between the first and second lumbar vertebrae. It is composed of a bundle of nerve fibers and associated tissues that connect the brain to nearly all parts of the body. The term “spinal cord tissue” refers to the bundle of nerve fibers and associated tissue which is joined together to create the spinal cord. The spinal cord has two basic types of tissue, gray matter and white matter. Gray matter makes up the core of the cord, has predominantly unmyelinated structures and is spatially organized with regard to neural circuitry. White matter is peripheral to each side of the spinal cord and comprises axons which are spatially organized into tracts. The cellular constituents of the spinal cord include neuron cell bodies, axons, glial cells, ependymal cells, vascular cells, and others.

As indicated, nucleic acid molecules of the present invention may be in the form of RNA, such as mRNA, are in the form of DNA, including, for instance, cDNA and genomic DNA obtained by cloning or produced synthetically. The DNA may be double-stranded or single-stranded. Single-stranded DNA or RNA may be the coding strand, also known as the sense strand, or it may be the non-coding strand, also referred to as the anti-sense strand.

The terms “DNA sequence encoding”, “DNA encoding” and “nucleic acid encoding” refer to the order or sequence of deoxyribonucleotides along a strand of deoxyribonucleic acid. The order of these deoxyribonucleotides determines the order of amino acids along the polypeptide chain. The DNA sequence thus codes for the amino acid sequence.

The term “control sequences” refers to DNA sequences necessary for the expression of an operably linked coding sequence in a particular host organism. The control sequences that are suitable for prokaryotes, for example, include a promoter, optionally an operator sequence, a ribosome binding site, and possibly, other as yet poorly understood sequences. Eukaryotic cells are known to utilize promoters, polyadenylation signals, and enhancers.

An “exogenous” element is defined herein to mean nucleic acid sequence that is foreign to the cell, or homologous to the cell but in a position within the host cell nucleic acid in which the element is ordinarily not found.

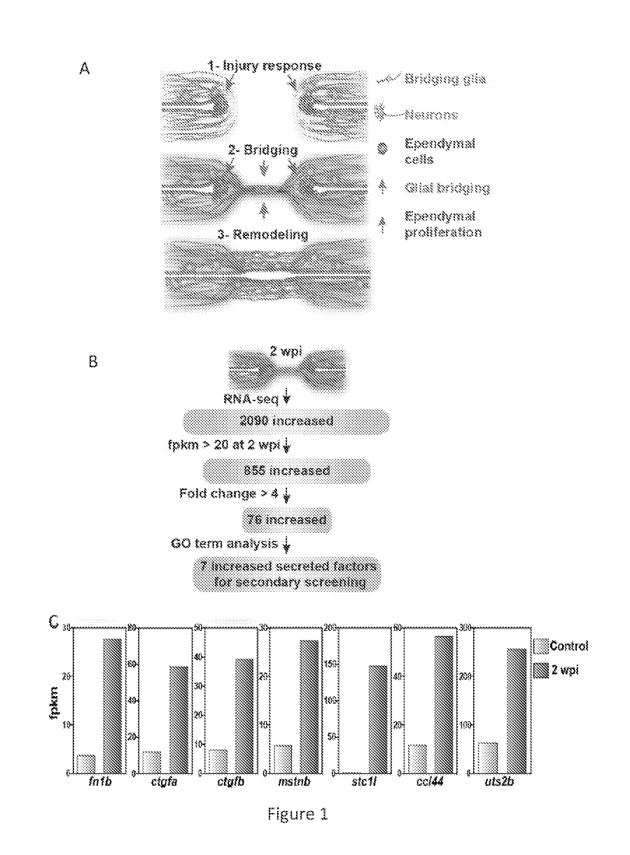

“High content screening (HCS)” was employed in the discovery of the present invention. A genome-wide profiling screen for secreted factors induced during zebrafish spinal cord regeneration was performed. Transcriptomes of injured and sham-injured SCs at 2 weeks post-injury (wpi) were profiled and 2090 genes with transcript levels that significantly increase after injury were identified (FIG. 1B). An expression level cutoff at 20 fragments per Kb per million mapped reads (fpkm) and an expression change cutoff at 4-fold was used to capture sharply induced factors. Seventy-six genes with increased expression met these criteria. Through Gene Ontology analysis, 7 of the 76 genes were classified as secreted, extracellular proteins (FIG. 1C).

As used herein, connective tissue growth factor (CTGF) is an extracellular, multimodular protein that contains four protein interaction domain. Further, as used herein the terms “protein” or “peptide” are used interchangeably, particularly with regard to CTGF, and as described in the claims. CTGF is involved in multiple cell processes, ranging from cell differentiation and fibrosis, to cell proliferation and tumorigenesis. But CTGF functions were not studied in the context of SC and brain regeneration. CTGF protein is highly conserved between zebrafish, mice, and humans as shown by protein alignment. By in situ hybridization, ctgf mRNA is not detectable in uninjured SC, but is strongly induced at 1wpi and 2wpi. Expression at 2wpi localizes to ventral ependymal cells and to the bridging area of the SC.

The terms “regenerate,” “regeneration,” and the like are used interchangeably to mean the restoration or new growth of tissue, that has been lost, removed, or injured. In certain embodiments, regeneration specifically includes ependymal cell proliferation at the lesion site in spinal cord tissue, extension of glial cells to connect the severed spinal cord tissue, growth of nerve axons across the lesion site, and recovery of motor functions that were lost after injury. A person having skill in the art is aware that this use coincides with the normal meaning of “regenerate” in the art.

As used herein, the phrase “initiating glial bridge formation” is used in describing a bridge of glial cells which connects the two severed SC portions. Goldshmit et al., J Neurosci 32, 7477-7492 (2012). Glial cells connect the transected SC and form a bridge along which axons will grow. Bridging is a striking, pro-regenerative glial response thought to provide a natural scaffold for axonal growth. Goldshmit et al., J Neurosci 32, 7477-7492 (2012). A person having skill in the art is aware that this use coincides with the normal meaning of “initiating glial bridge formation” in the art.

As used herein, the phrase “stimulating axon tracts” is used to describe the formation/generation of axon tracts or axonal growth in the spinal cord tissue. In some embodiments, the formation of the axon tracts occurs at a newly formed glial bridge. A person having skill in the art is aware that this use coincides with the normal meaning of “stimulating axon tracts” in the art.

As used herein, the phrase “inducing neuron formation” is used in describing the making, formation, and creation of neurons “neurogenesis.” In some embodiments, the neurogenesis occurs in the spinal cord tissue at the point of injury. A person having skill in the art is aware that this use coincides with the normal meaning of “inducing neuron formation” in the art.

In the context of the present disclosure the expressions “cell”, “cell line”, “cell model,” and “cell culture” are used interchangeably, and all such designations include progeny. This includes the primary subject cell, either established from a transgenic animal or created in the laboratory, and cultures derived therefrom without regard for the number of transfers. It is also understood that all progeny may not be precisely identical in DNA content, due to deliberate or inadvertent mutations. Mutant progeny that have the same function or biological activity as screened for in the originally transformed cell are included. Where distinct designations are intended, it will be clear from the context.

“Animal model,” “mouse model,” “knock-out model,” and “transgenic animal” are terms used interchangeably and all such terms are used to describe animals that have had an exogenous element deliberately inserted into or removed from their genome. Such animals are most commonly created by the micro-injection of DNA into the pronuclei of a fertilized egg which is subsequently implanted into the oviduct of a pseudopregnant surrogate mother. These such designations also include the primary subject animal and progeny derived therefrom without regard for the number of progeny and generations.

As used herein, “modified zebrafish” are used as an animal model. The modified zebrafish are animals that have had an exogenous element deliberately inserted into or an endogenous element removed from their genome. In order to produce a genetically modified zebrafish, a genomic sequence upstream of the target gene translational start site is placed upstream of a protein reporter cassette. Specific primers are used to amplify the genomic DNA region upstream of the target gene translational start site. The produced genomic fragment is cloned into a vector, then subcloned into a digested plasmid. The genetically modified construct is injected into one-cell stage wild-type embryos. Founders are then isolated and propagated.

The term “administering” or “administered” as used herein is meant to include both parenteral and/or oral administration, all of which are described in more detail in the “pharmaceutical compositions” section below. By “parenteral” is meant intravenous, subcutaneous or intramuscular administration. In the methods of the subject disclosure, the interfering molecules of the present disclosure may be administered alone, simultaneously with one or more other interfering molecule, or the compounds may be administered sequentially, in either order. It will be appreciated that the actual preferred method and order of administration will vary according to, inter alia, the particular preparation of interfering molecules being utilized, the particular formulation(s) of the one or more other interfering molecules being utilized. The optimal method and order of administration of the compounds of the disclosure for a given set of conditions can be ascertained by those skilled in the art using conventional techniques and in view of the information set out herein. The term “administering” or “administered” also refers to oral sublingual, buccal, transnasal, transdermal, rectal, intramuscular, intravenous, intraventricular, intrathecal, and subcutaneous routes. In accordance with good clinical practice, it is preferred to administer the instant compounds at a concentration level which will produce effective beneficial effects without causing any harmful or untoward side effects.

According to the present disclosure, a “therapeutically effective amount” of a pharmaceutical composition is an amount which is sufficient for the desired pharmacological effect. The amount that is “effective” will vary from subject to subject, depending on the age and general condition of the individual, mode of administration, and the like. Thus, it is not always possible to specify an exact “effective amount.” However, an appropriate “effective” amount in any individual case may be determined by one of ordinary skill in the art using routine experimentation.

As used herein, the term “ameliorate” refers to the ability to make better, or more tolerable, or reduce, the clinical characterization of mammalian spinal cord injury. The term “treating” refers to the caring for, or dealing with, a subject's spinal cord injury condition either medically or surgically, and can include “ameliorating” and/or “limiting progression.” Also within the scope of the term “treating” is the acting upon a subject presenting the clinical features of spinal cord injury by the use of some agent, such as an interfering molecule, to amelioriate, improve, alter, or reduce the condition.

As used herein, the term “pharmaceutical composition” means physically discrete coherent portions suitable for medical administration. The term “dosage unit form” or “unit dosage” means physically discrete coherent units suitable for medical administration, each containing a daily dose or a multiple (up to four times) or a sub-multiple (down to a fortieth) of a daily dose of the active compound in association with a carrier and/or enclosed within an envelope. Whether the composition contains a daily dose, or for example, a half, a third or a quarter of a daily dose, will depend on whether the pharmaceutical composition is to be administered once or, for example, twice, three times or four times a day, respectively.

The proteins and more particularly the CTGF protein of the present disclosure may be administered to the subject as a composition which comprises a pharmaceutically effective amount of protein and an acceptable carrier and/or excipients. A pharmaceutically acceptable carrier includes any solvents, dispersion media, or coatings that are physiologically compatible. Preferably, the carrier is suitable for intravenous, intramuscular, oral, intraperitoneal, intradermal, transdermal, topical, nasal or subcutaneous administration. One exemplary pharmaceutically acceptable carrier is physiological saline.

Other pharmaceutically acceptable carriers and their formulations are well-known and generally described in, for example, Remington's Pharmaceutical Science (18^{th} Ed., ed. Gennaro, Mack Publishing Co., Easton, Pa., 1990). Various pharmaceutically acceptable excipients are well-known in the art and can be found in, for example, Handbook of Pharmaceutical Excipients (4^{th} ed., Ed. Rowe et al. Pharmaceutical Press, Washington, D.C.). The composition can be formulated as a solution, microemulsion, liposome, capsule, tablet, or other suitable forms.

In embodiments, the active component which comprises a protein of the present invention may be coated in a material to protect it from inactivation by the environment prior to reaching the target site of action. The pharmaceutical compositions of the present disclosure are preferably sterile and non-pyrogenic at the time of delivery, and are preferably stable under the conditions of manufacture and storage.

In other embodiments of the present disclosure, the pharmaceutical compositions are regulated-release formulations. Proteins of the present invention and particularly CTGF proteins may be admixed with biologically compatible polymers or matrices which control the release rate of the copolymers into the immediate environment. Controlled or sustained release compositions include formulation in lipophilic depots (e.g., fatty acids, waxes, oils).

In some embodiments of the present disclosure, pharmaceutical compositions comprise peptides formulated with oil and emulsifier to form water-in-oil microparticles and/or emulsions. The oil may be any non-toxic hydrophobic material liquid at ambient temperature to about body temperature, such as edible vegetable oils including safflower oil, soybean oil, corn oil, and canola oil; or mineral oil. Chemically defined oil substance such as lauryl glycol may also be used. The emulsifier useful for this embodiment includes Span 20 (sorbitan monolaurate) and phosphatidylcholine.

In some embodiments, a peptide's composition is prepared as an aqueous solution and is prepared into a water-in-oil emulsion dispersed in 95 to 65% oil such as mineral oil, and 5 to 35% emulsifier such as Span 20. In another embodiment of the disclosure, the emulsion is formed with alum rather than with oil and emulsifier. These emulsions and microparticles reduce the speed of uptake of peptides, and achieve controlled delivery. In other embodiments, the pharmaceutical compositions also include additional therapeutically active agents.

The present disclosure further provides a kit comprising (i) a composition comprising a peptide and (ii) instructions for administering the composition to a subject in need thereof at intervals greater than 24 hours, more preferably greater than 36 hours, for the treatment of injury to the nervous system and spinal cord. In an embodiment, the peptide encodes CTGF. In one embodiment, the peptide is formulated in dosages for administration multiple times daily including hourly, every 2 hours, three hours, four hours, six hours, eight hours, or twice daily including 12 hours, or any intervening interval thereof. In another embodiment, the peptide is formulated in dosages for administration of greater than about 24, 30, 36, 42, 48, 54, 60, 66, 72, 78, 84, 90, 96, 102, 108, 114, 120, 126, 132, 138, 144, 150, 156, 162, 168, 174, 180, 186, 192, 198, 204, 210, 216, 222, 228, 234, or 240 hours, or any intervening interval thereof. In another embodiment of the kits described herein, the instructions indicate that the peptide is to be administered about every 24, 30, 36, 42, 48, 54, 60, 66, 72, 78, 84, 90, 96, 102, 108, 114, 120, 126, 132, 138, 144, 150, 156, 162, 168, 174, 180, 186, 192, 198, 204, 210, 216, 222, 228, 234, or 240 hours, or any interval in between. Kits may comprise additional components, such as packaging and one or more apparatuses for the administration of the peptide, such as a hypodermic syringe.

In some embodiments of the present disclosure, a suitable dose of a therapeutic peptide composition is administered that will be the lowest effective dose to produce a therapeutic effect, for example, mitigating symptoms. In certain embodiments, the therapeutic peptides are administered at a dose per subject, which corresponds to a dose per day of at least about 2 mg, at least about 5 mg, at least about 10 mg, or at least about 20 mg as appropriate minimal starting dosages, or about x mg, wherein x is an integer between 1 and 20. In one embodiment of the methods described herein, a dose of about 0.01 to about 500 mg/kg can be administered. In general, the effective dosage of the compound of the present disclosure can readily be determined as routine practice by one of skill in the art. Thus the embodiments above are not meant to be a limiting, but merely representative dosage examples.

However, it is understood by one skilled in the art that the dose of the composition of the present disclosure will vary depending on the subject and upon the particular route of administration used. It is routine in the art to adjust the dosage to suit the individual subjects. Additionally, the effective amount may be based upon, among other things, the size of the compound, the biodegradability of the compound, the bioactivity of the compound, and the bioavailability of the compound. For example, if the compound does not degrade quickly, is bioavailable and highly active, a smaller amount will be required to be effective. The actual dosage suitable for a subject can easily be determined as a routine practice by one skilled in the art, for example a physician or a veterinarian given a general starting point. For example, the physician or veterinarian could start doses of the compound of the invention employed in the pharmaceutical composition at a level lower than that required in order to achieve the desired therapeutic effect, and increase the dosage with time until the desired effect is achieved.

In the context of the present disclosure, the term “treatment regimen” is meant to encompass therapeutic, palliative and prophylactic modalities of administration of one or more compositions comprising one or more peptides. A particular treatment regimen may last for a period of time which will vary depending upon the nature of the particular injury, its severity, and the overall condition of the patient, and the treatment regimen may extend from multiple daily doses, once daily, or more preferably once every 36 hours or 48 hours or longer, to once every month or several months. Following treatment, the patient is monitored for changes in his/her condition and for alleviation of the symptoms of the injury or disease state. The dosage of the peptides may either be increased in the event the patient does not respond significantly to current dosage levels, or the dose may be decreased if an alleviation of the symptoms of the injury or disease state is observed, or if the injury or disease state has been ablated, or if unacceptable side effects are seen with the starting dosage.

In one embodiment, a therapeutically effective amount of the peptide is administered to the subject in a treatment regimen comprising intervals of at least 36 hours, or more preferably 48 hours, between dosages. In another embodiment, the peptide is administered at intervals of at least 54, 60, 66, 72, 78, 84, 90, 96, 102, 108, 114, 120, 126, 132, 138, 144, 150, 156, 162, 168, 174, 180, 186, 192, 198, 204, 210, 216, 222, 228, 234, or 240 hours, or the equivalent amount of days. In some embodiments, the agent is administered every other day, while in other embodiments it is administered weekly. If two peptides are administered to the subject, such peptides may be administered at the same time, such as simultaneously, or essentially at the same time, such as in succession. Alternatively, their administration may be staggered. For example, two peptides which are each administered every 48 hours may both be administered on the same days, or one may be administered one day and the other on the next day and so on in an alternating fashion.

In other embodiments, the peptide is administered in a treatment regimen which comprises at least one uneven time interval, wherein at least one of the time intervals is at least 24, 30, 36, 42, 48, 54, 60, 66, 72, 78, 84, 90, 96, 102, 108, 114, 120, 126, 132, 138, 144, 150, 156, 162, 168, 174, 180, 186, 192, 198, 204, 210, 216, 222, 228, 234, or 240 hours, or the equivalent amount of days.

In one embodiment, the peptide is administered to be subject at least three times during a treatment regimen, such that there are at least two time intervals between administrations. These intervals may be denoted I₁ and I₂. If the peptide is administered four times, then there would be an additional interval between the third and fourth administrations, I₃, such that the number of intervals for a given number “n” of administrations is n−1. In one embodiment, the interval for administration is multiple times daily including hourly, every 2 hours, three hours, four hours, six hours, eight hours, or twice daily including 12 hours, or any intervening interval thereof. Accordingly, in one embodiment, at least one of the time intervals between administrations is greater than about 24, 30, 36, 42, 48, 54, 60, 66, 72, 78, 84, 90, 96, 102, 108, 114, 120, 126, 132, 138, 144, 150, 156, 162, 168, 174, 180, 186, 192, 198, 204, 210, 216, 222, 228, 234, or 240 hours. In another embodiment, at least 1%, 2%, 3%, 4%, 5%, 10%, 15%, 20%, 25%, 30%, 40%, 50%, 60%, 70%, 80%, 90% or 95% of the total number n−1 of time intervals are at least about 2, 4, 6, 8, 10, 12, 24, 30, 36, 42, 48, 54, 60, 66, 72, 78, 84, 90, 96, 102, 108, 114, 120, 126, 132, 138, 144, 150, 156, 162, 168, 174, 180, 186, 192, 198, 204, 210, 216, 222, 228, 234, or 240 hours.

In yet another embodiment, the average time interval between administrations ((I₁+I₂+ . . . +Iₙ₋₁)/n−1) is at least 2, 4, 6, 8, 10, 12, 24, 30, 36, 42, 48, 54, 60, 66, 72, 78, 84, 90, 96, 102, 108, 114, 120, 126, 132, 138, 144, 150, 156, 162, 168, 174, 180, 186, 192, 198, 204, 210, 216, 222, 228, 234, or 240 hours, or at least two weeks.

In another embodiment, the dosage regimen consists of two or more different interval sets. For example, a first part of the dosage regimen is administered to a subject multiple daily, daily, every other day, or every third day, for example, at about 22 mg peptide/m² body surface area of the subject, wherein the subject is a human. In some embodiments of the invention, the dosing regimen starts with dosing the subject every other day, every third day, weekly, biweekly, or monthly. The dosage for administration every other day or every third day may be up to about 65 mg/m² and 110 mg/m² respectively. For a dosing regimen comprising dosing of the peptide every week, the dose comprises up to about 500 mg/m², and for a dosing regimen comprising dosing of the peptide every two weeks or every month, up to 1.5 g/m² may be administered. The first part of the dosing regimen may be administered for up to 30 days, for example, 7, 14, 21, or 30 days. A subsequent second part of the dosing regimen with a different, longer interval administration with usually lower exposure (step-down dosage), administered weekly, every 14 days, or monthly may optionally follow, for example, at 500 mg/m² body surface area weekly, up to maximum of about 1.5 g/m² body surface area, continuing for 4 weeks up to two years, for example, 4, 6, 8, 12, 16, 26, 32, 40, 52, 63, 68, 78, or 104 weeks. Alternatively, if the injury of the nervous system heals or generally improves, the dosage may be maintained or kept at lower than maximum amount, for example, at 140 mg/m² body surface area weekly. If, during the step-down dosage regimen, the injury condition relapses, the first dosage regimen may be resumed until effect is seen, and the second dosing regimen may be implemented. This cycle may be repeated multiple times as necessary.

More specifically, one aspect of the disclosure is treatment of disorders of the nervous system treatable with a peptide, such as injuries to the spinal cord. One embodiment of the disclosure is a method for treating disorders of the nervous system treatable with peptides of the composition (SEQ ID NO: 15) in a molar input ratio of about 1.0:1.0:10.0:6.0 respectively, synthesized by solid phase chemistry, wherein the copolymer has a length of 25 amino acids, by administering the peptide to a human subject in need of treatment a first part of a dosing regimen comprising a dose of about 22 mg/m² body surface area daily. In some embodiment of the disclosure, the dosing regimen starts with dosing the subject every other day, every third day, weekly, biweekly, or monthly. The dosage for administration every other day or every third day may be up to about 65 mg/m² and 110 mg/m² respectively. For a dosing regimen comprising dosing of the peptide every week, the dose comprises up to about 500 mg/m², and for a dosing regimen comprising dosing of the peptide every two weeks or every month, up to 1.5 g/m² may be administered. The first part of the dosing regimen may be administered for up to 30 days, for example, 7, 14, 21, or 30 days. A subsequent second part of the dosing regimen with a different, longer interval administration with usually lower exposure (step-down dosage), administered weekly, every 14 days, or monthly may optionally follow, for example, at 500 mg/m² body surface area weekly, up to maximum of about 1.5 g/m² body surface area, continuing for 4 weeks up to two years, for example, 4, 6, 8, 12, 16, 26, 32, 40, 52, 63, 68, 78, or 104 weeks. Alternatively, if the condition heals or generally improves, the dosage may be maintained or kept at lower than maximum amount, for example, at 140 mg/m² body surface area weekly. If, during the step-down dosage regimen, the condition relapses, the first dosage regimen may be resumed until effect is seen, and the second dosing regimen may be implemented. This cycle may be repeated multiple times as necessary.

Any of the methods and means may be practiced using compositions and formulations described in this application.

In other embodiments of the present disclosure, any of the methods of the disclosure may be practiced using sustained release formulation comprising a peptide. When administering a peptide of the disclosure using a sustained release formula, the overall exposure to the peptide is generally lower than in bolus administration. For example, a first part of the dosage regimen is administered to a subject daily, every other day, or every third day, for example, at about 22 mg phosphopeptide/m² body surface area of the subject, wherein the subject is a human.

In some embodiment of the present disclosure, the dosing regimen uses sustained release formula, dosing the subject every other day, every third day, weekly, biweekly, or monthly so that the phosphopeptide is released during the interval. The dosage for administration every other day or every third day may be up to about 35 mg/m² and 65 mg/m² respectively. For a dosing regimen comprising dosing of the phosphopeptide every week, the dose comprises up to about 140 mg/m², and for a dosing regimen comprising dosing of the phosphopeptide every two weeks or every month, up to 750 mg/m² may be administered. The first part of the dosing regimen may be administered for up to 30 days, for example, 7, 14, 21, or 30 days. A subsequent second part of the dosing regimen with a different, longer interval administration with usually lower exposure (step-down dosage), administered weekly, every 14 days, or monthly may optionally follow, for example, at 140 mg/m² body surface area weekly, up to maximum of about 1.5 g/m² body surface area, continuing for 4 weeks up to two years, for example, 4, 6, 8, 12, 16, 26, 32, 40, 52, 63, 68, 78, or 104 weeks. Alternatively, if the disease goes into remission or generally improves, the dosage may be maintained or kept at lower than maximum amount, for example, at 140 mg/m² body surface area weekly. If, during the step-down dosage regimen, the disease condition relapses, the first dosage regimen may be resumed until effect is seen, and the second dosing regimen may be implemented. This cycle may be repeated multiple times as necessary.

In certain embodiments of the methods described herein, the route of administration can be oral, intraperitoneal, transdermal, subcutaneous, by intravenous or intramuscular injection, by inhalation, topical, intralesional, infusion; liposome-mediated delivery; topical, intrathecal, gingival pocket, rectal, intravaginal, intrabronchial, nasal, transmucosal, intestinal, ocular or otic delivery, or any other methods known in the art as one skilled in the art may easily perceive. Other embodiments of the compositions of the present disclosure incorporate particulate forms protective coatings, protease inhibitors or permeation enhancers for various routes of administration, including parenteral, pulmonary, nasal and oral. Administration can be systemic or local. In a preferred embodiment, the peptide is administered subcutaneously.

An embodiment of the methods of present disclosure relates to the administration of the copolymers of the present invention in a sustained release form. Such method comprises applying a sustained-release transdermal patch or implanting a sustained-release capsule or a coated implantable medical device so that a therapeutically effective dose of the peptide of the present disclosure is delivered at defined time intervals to a subject of such a method. The compounds and/or agents of the subject disclosure may be delivered via a capsule which allows regulated-release of the phosphopeptide over a period of time. Controlled or sustained-release compositions include formulation in lipophilic depots (e.g., fatty acids, waxes, oils). Also comprehended by the invention are particulate compositions coated with polymers (e.g., poloxamers or poloxamines).

For oral administration, the pharmaceutical preparation may be in liquid form, for example, solutions, syrups or suspensions, or may be presented as a drug product for reconstitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, or fractionated vegetable oils); and preservatives (e.g., methyl or propyl-p-hydroxybenzoates or sorbic acid). The pharmaceutical compositions may take the form of, for example, tablets or capsules prepared by conventional means with pharmaceutically acceptable excipients such as binding agents (e.g., pre-gelatinized maize starch, polyvinyl pyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulfate). The tablets may be coated by methods well-known in the art.

When the peptide is introduced orally, it may be mixed with other food forms and consumed in solid, semi-solid, suspension, or emulsion form; and it may be mixed with pharmaceutically acceptable carriers, including water, suspending agents, emulsifying agents, flavor enhancers, and the like. In one embodiment, the oral composition is enterically-coated. Use of enteric coatings is well known in the art. For example, Lehman (1971) teaches enteric coatings such as Eudragit S and Eudragit L. The Handbook of Pharmaceutical Excipients, 2.sup.nd Ed., also teaches Eudragit S and Eudragit L applications. Preparations for oral administration may be suitably formulated to give controlled release of the active compound.

For buccal administration, the compositions may take the form of tablets or lozenges formulated in conventional manner. The compositions may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen free water, before use.

The compositions may also be formulated in compositions for administration via inhalation. For such administration, the compositions for use according to the present disclosure are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of, e.g., gelatin, for use in an inhaler or insufflator may be formulated containing a powder mix of the compound and a suitable powder base such as lactose or starch.

In certain embodiments, compositions comprising the peptide of the present disclosure are formulated in accordance with routine procedures as pharmaceutical compositions adapted for intravenous administration to human beings. Typically, compositions for intravenous administration are solutions in sterile isotonic aqueous buffer. Where necessary, the composition may also include a solubilizing agent and a local anesthetic such as lignocaine to ease pain at the site of the injection. Generally, the ingredients are supplied either separately or mixed together. Where the composition is to be administered by infusion, it can be dispensed with an infusion bottle containing sterile pharmaceutical grade water or saline, with the intervals between administrations being greater than 24 hours, 32 hours, or more preferably greater than 36 or 48 hours. Where the composition is administered by injection, an ampoule of sterile water or saline for injection can be provided so that the ingredients may be mixed prior to administration.

In certain embodiments, the methods described herein allow continuous treatment of injuries and conditions of the nervous system by a sustained-release carrier such as transdermal patches, implantable medical devices coated with sustained-release formulations, or implantable or injectable pharmaceutical formulation suitable for sustained-release of the active components. In such embodiments, the intervals between administrations are preferably greater than 24 hours, 32 hours, or more preferably greater than 36 or 48 hours. For instance, an implantable device or a sustained released formulation which releases the peptide over a 2 day period may be implanted every four days into the patient, such that the interval during which no peptide is administered to the subject is 2 days. In related embodiments, the such interval where during which no administration occurs is at least 24+x hours, wherein x represents any positive integer.

In another embodiment, the peptides are formulated to have a therapeutic effect when administered to a subject in need thereof at time intervals of at least 24 hours. In a specific embodiment, the peptides are formulated for a long-lasting therapeutic affect such that a therapeutic effect in treating the disease is observed when the peptides are administered to the subject at time intervals of at least 24, 30, 36, 42, 48, 54, 60, 66, 72, 78, 84, 90, 96, 102, 108, 114, 120, 126, 132, 138, 144, 150, 156, 162, 168, 174, 180, 186, 192, 198, 204, 210, 216, 222, 228, 234, or 240 hours between administrations.

In other embodiments of the methods described herein, additional therapeutically active agents are administered to the subject. In one embodiment, compositions comprising additional therapeutic agents(s) are administered to the subject as separate compositions from those comprising the peptide. For example, a subject may be administered a composition comprising a peptide subcutaneously while a composition comprising another therapeutic agent may be administered orally. The additional therapeutically active agents may treat the same disease as the peptide, a related disease, or may be intended to treat an undesirable side effect of administration of the copolymer, such as to reduce swelling at a site of intradermal injection.

In some aspects, the present disclosure further provides a method of regenerating or repairing neural tissue in a subject in need thereof, the method comprising administering a population of cells enriched for the expression of one or more of ctgf and gfap protein or mRNA. In embodiments, the present disclosure provides a method of regenerating or repairing an injury to the spinal cord in a subject in need thereof comprising administering a population of cells enriched for the expression of one or more of ctgf and gfap protein or mRNA.

In other embodiments, the present disclosure provides a method of regenerating or repairing neural tissue in a subject with a neurodegenerative condition comprising administering a population of cells enriched for the expression of one or more of ctgf and gfap protein or mRNA. In still further embodiments, the present disclosure provides a method of regenerating or repairing neural tissue in a subject with a neurodevelopmental condition comprising administering a population of cells enriched for the expression of one or more of ctgf and gfap protein or mRNA.

In embodiments, the disclosure provides a composition comprising a population of cells expressing one or more of ctgf and gfap positive cells useful for administration to a subject in need of spinal cord regeneration or repair. In certain embodiments, the disclosure provides a purified population of cells expressing one or more of ctgf and gfap positive cells prepared by cell sorting, for example fluorescence activated cell sorting. In other embodiments, the disclosure provides a population of cells expressing one or more of ctgf and gfap positive cells prepared by overexpressing one or more of ctgf and gfap protein and/or mRNA. In alternative embodiments, a combination of methods is employed to generate a population of cells expressing one or more of ctgf and gfap positive cells.

In another aspect, the present disclosure provides a method of identifying and/or isolating glial cells useful for the repair or regeneration of injury in a subject in need thereof, comprising detecting a level of ctgf protein or mRNA expression in a glial cells, and selecting the glial cells expressing ctgf protein or mRNA.

In yet another aspect, the present disclosure provides a method of engineering glial cells with desirable properties comprising expressing ctgf protein or mRNA in said glial cells. In some embodiments, expressing ctgf protein or mRNA provides engineered glial cells with improved axon growth-permissive potential. In some embodiments, expressing ctgf protein or mRNA provides glial cells with improved axon briding potential. In still further embodiments, expressing ctgf protein or mRNA provides glial cells with any of the therapeutic or prophylactic properties disclosed herein.

Methods well known to those skilled in the art can be used to practice any of the embodiments of the present disclosure. See, for example, techniques as described in Maniatis et al., 1989, MOLECULAR CLONING: A LABORATORY MANUAL, Cold Spring Harbor Laboratory, New York; Ausubel et al., 1989, CURRENT PROTOCOLS IN MOLECULAR BIOLOGY, Greene Publishing Associates and Wiley Interscience, New York; Sambrook, J. et al., 2001, “MOLECULAR CLONING: A LABORATORY MANUAL,” 3.sup.rd edition, Cold Spring Harbor Laboratory Press. The contents of the above are incorporated in their entirety herein by reference.

Additional methods well known to those skilled in the art can be used to prepare pharmaceutically acceptable compositions and methods of treatment according to the present disclosure. See, for example, Goodman & Gilman, 2005, THE PHARMACOLOGICAL BASIS OF THERAPEUTICS,” 11.sup.th Edition, McGraw-Hill. The contents of the above are incorporated in their entirety herein by reference.

Additional methods well known to those skilled in the art can be used for therapeutic intervention in subjects with metabolic dysfunction. (See e.g. Physician's Desk Reference, Medical Economics Company, Inc. Montvale, N.J. (54th Edition) 2000; American Association of Clinical Endocrinologists Medical Guidelines for Clinical Practice for Growth Hormone use in Adults and Children-2003 Update, AACE Growth Hormone Task Force, Endocrine Practice (2003), 9:65-76.)

The invention will be further described in the following examples, which do not limit the scope of the invention described in the claims.

The Examples that follow are illustrative of specific embodiments of the invention and various uses thereof. They are set forth for explanatory purposes only and are not to be taken as limiting the invention.

### EXAMPLES

### Example 1 Transcriptome Profiling Indicates CTGFa is Upregulated Following Spinal Cord Injury

To identify potential genes with modulated expression following spinal cord injury, the following study and screen were performed.

Zebrafish Husbandry.

Adult zebrafish of the Ekkwill strain are maintained according to methods known in the art (K. D. Poss, et al. (2002) Science 298, 2188-2190; (Westerfield, M. (2000) The Zebrafish Book-A guide for the laboratory use of zebrafish (Danio rerio), University of Oregon Press, Eugene, Oreg.) Animals between 6 and 12 months of both sexes are used. All surgeries, histological analyses, and proliferation analyses are performed in a blinded fashion, and experiments are repeated using different clutches of animals. All procedures using animals are approved by the Institutional Animal Care and Use Committee at Duke University. Newly constructed strains are described below.

Spinal Cord Transection and Treatments.

Zebrafish were anaesthetized using MS-222. Fine scissors were used to make a small incision that transects the spinal cord 4 mm caudal to the brainstem region. Complete transection was visually confirmed at the time of surgery. Injured animals were then assessed using a swim tunnel (Loligo, catalog #SW100605L, 120V/60 Hz) at 2 or 3 dpi to confirm loss of swim capacity after surgery.

RNA Sequencing.

Two mm SC sections, including the lesion site plus additional rostral tissue proximal to the lesion, are collected from adult injured zebrafish at 2 wpi. Control tissue sections are collected from sham-injured clutchmate animals. Total RNA is prepared using Tri reagent (Sigma). TruSeq libraries are prepared in duplicate and sequenced on Illumina HiSeq 2000 using 50 bp single-end reading strategy. Quality QC and trimming of adapters and short sequences are performed using Fastx. Sequencing reads are then mapped to the zebrafish genome (Zv9) using Bowtie2, then assembled and quantified using the Cufflinks and Cuffdiff algorithms (C. Trapnell et al. (2010) Nat Biotechnol 28, 511-515). GO analysis is performed using the DAVID 6.7 bioinformatics resources (G. Dennis, Jr. et al. (2003) Genome Biol 4, P3). The GEO accession number for this dataset is GSE77025.

Results of Transcriptome Analysis.

The transcriptomes of injured and sham-injured SCs are profiled at 2 weeks post-injury (wpi). 2090 genes are identified with transcript levels that significantly increase after injury (FIGS. 1B and C). To capture sharply induced factors, an expression level cutoff is set at 20 fragments per Kb per million mapped reads (fpkm), and an expression change cutoff is set at 4-fold. Seventy-six genes with increased expression meet these criteria (i.e., 76 genes demonstrated significantly increased expression post-injury). By Gene Ontology analysis, 7 of the 76 genes are classified as secreted, extracellular proteins (FIG. 1B), including fibronectin, a known (i.e. control) axon growth-promoting extracellular molecule that validates the results of the transcriptome screen (V. J. Tom, et al. (2004) J Neurosci 24, 9282-9290; C. Y. Lin et al. (2012) J Neurotrauma 29, 589-599.) The other six genes demonstrating increased expression following spinal cord injury include: ctgfa, ctgfb, myostatin b (mstnb), stanniocalcin 1 like (stc1l), ccl44, and uts2b (FIG. 1C).

### Example 2: Histologic and Molecular Genetic Analyses Show the Spatiotemporal Pattern of ctgfa Expression Correlates with Glial Bridge Formation

CTGF elicits various cellular responses including adhesion, migration, proliferation, and differentiation. CTGF expression is induced after rat CNS injury (M. Hertel, et al. (2000) Eur J Neurosci 12, 376-380; Y. Liu et al. (2014) Diagn Pathol 9, 141; S. Conrad, et al. (2005) J Neurosurg Spine 2, 319-326). The function of CTGF in the vertebrate CNS are unknown to the art. As ctgfa expression is induced in the injured zebrafish SC (Example 1, supra), this and the subsequent Examples investigate and demonstrate CTGF's pro-regenerative functions in vertebrate CNS.

Histology.

In situ hybridization is performed using 16 μm (transverse) or 20 μm (longitudinal) cryosections of paraformaldehyde-fixed SCs. In situ hybridization probes for ctgfa are subcloned after amplification from 2 dpf zebrafish cDNA into either PCRII-Blunt-TOPO or PCR2.1-TOPO vectors (ctgfa forward primer 5′-tgtgattgctctgctgttcc-3′(SEQ ID NO: 1), ctgfa reverse primer 5′-ggtgaggcgattagcttctg-3′ (SEQ ID NO: 2)). Linearized vectors are used to generate the digoxygenin-labeled cRNA probes. In situ hybridizations are performed with the aid of an InSituPro robot (Intavis) as described (K. D. Poss, et al. (2002) Development 129, 5141-5149). Primary antibodies used in these examples are rabbit anti-GFAP (Sigma, G9269, 1:1000), mouse anti-GFAP (ZIRC, Zrf1, 1:1000) and mouse anti-acetyl-α-tubulin (Sigma, T6793, 1:1000). Secondary antibodies (Invitrogen, 1:200) used in these examples are Alexa Fluor 488, Alexa Fluor 594 goat anti-rabbit or anti-mouse antibodies. SC tissues were imaged using a Leica DM6000 compound microscope (for in situ hybridization) or a Zeiss LSM 700 confocal microscope (for immunofluorescence).

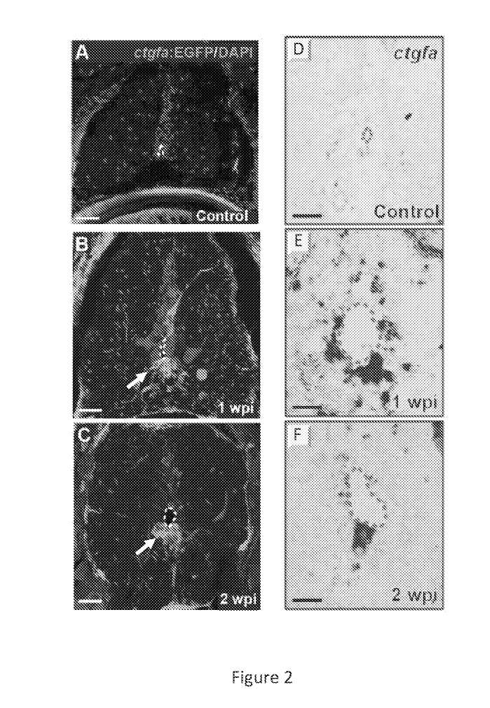

In situ hybridization assays are performed to visualize the expression of ctgfa before and after injury. Transcripts for ctgfa is minimally detected or absent from the uninjured SC, and are induced at 1 and 2 wpi in the ventral region of the ependyma (FIG. 2D-F).

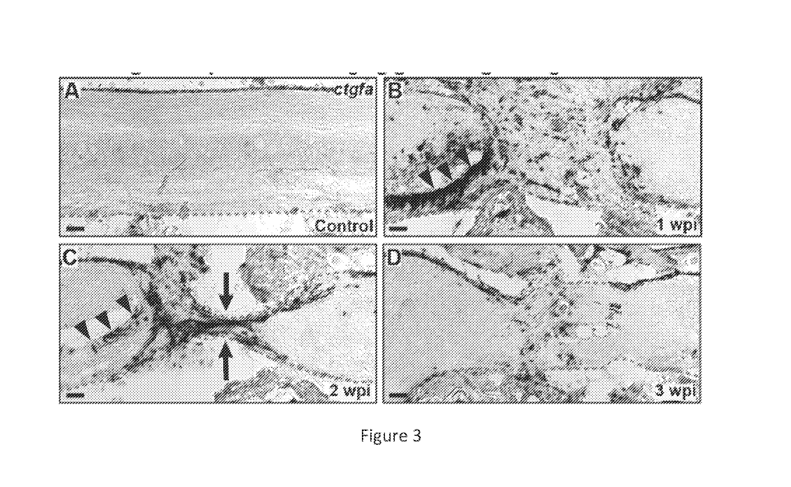

To further characterize ctgfa expression along the rostrocaudal SC axis, in situ hybridization is performed on longitudinal sections following SC injury (FIG. 3, A-D). At 1 wpi, ctgfa transcription is induced in multiple cell types across the lesion site and in ependymal cells at the central canal near the lesion (FIG. 3B). At this time point the strongest ctgfa signal is in the ventral ependyma. By 2 wpi, ctgfa expression is localized to ventral ependymal cells and marked the cellular bridge that had formed at the lesion site (FIG. 3C).

Generation of ctgfa:EGFP Zebrafish.

Transgenic reporter zebrafish (ctgfa:EGFP) are generated with a 5.5 kb genomic sequence upstream of the ctgfa translational start site placed upstream of a EGFP reporter cassette. The following primers are used to amplify a 5.5 kb genomic DNA region upstream of ctgfa translational start site: ClaI forward primer 5′-atcgattttggctactttcagctaagactgg-3′ (SEQ ID NO: 3) and ClaI reverse primer 5′-atcgattctttaaagtttgtagcaaaaagaaa-3′ (SEQ ID NO: 4). The genomic fragment is cloned into PCR2.1-TOPO vector, then subcloned into ClaI digested PCS2-EGFP plasmid. The ctgfa:EGFP construct is co-injected into one-cell stage wild-type embryos with I-SceI. Three founders are isolated and propagated.

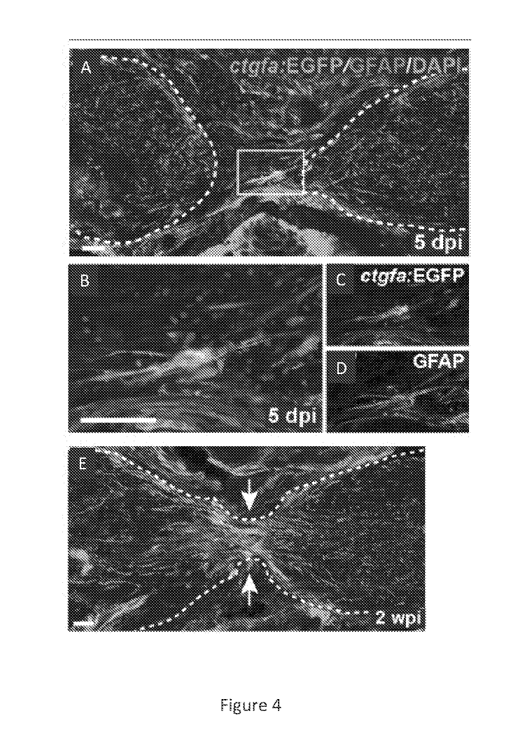

The ctgfa:EGFP reporter recapitulates endogenous mRNA expression in SC tissue at 1 and 2 wpi (FIG. 2, A-C). As early as 5 days post-injury (dpi), domains of ctgfa:EGFP and GFAP, a marker of multiple glial and radial glial cells in the CNS, overlap within a subpopulation of glial cells at the injury site marking the earliest bridging cells (FIG. 4A-D). Similarly, ctgfa:EGFP reporter expression colocalizes with the GFAP⁺ bridge at 2 wpi (FIG. 4E).

ctgfa expression is not detected in uninjured SC (FIG. 4A), but is strongly induced in ventral ependymal cells and around the lesion site at 1 wpi (FIG. 4B). At 2 wpi, ctgfa transcripts localize to ventral ependymal cells (arrowheads) and to the cellular bridge connecting the transected SC (arrows) (FIG. 4C). ctgfa expression is then diminished at 3 wpi (FIG. 4D). ctgfa-driven expression in early bridging glia suggests demarcation of a “pioneer” cell population responsible for repairing spinal cord injury.

### Example 3: A ctgfa Mutant Shows Impaired SC Regeneration

Generation of ctgfa^{bns50} Mutant Zebrafish.

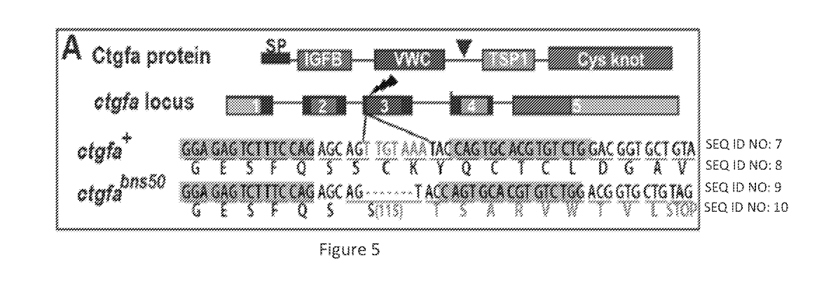

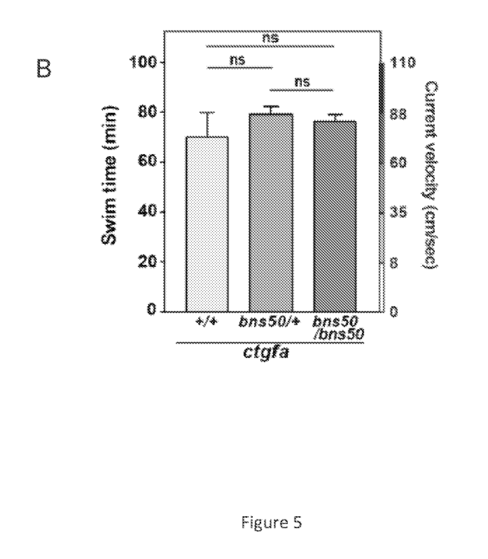

A ctgfa mutant allele (ctgfa^{bns50}) that harbors a frameshift-causing, 7 nt deletion within the third exon of the ctgfa locus (FIG. 5A), is used to determine if ctgfa is required for SC regeneration. TALEN constructs for left and right arms are designed to target exon 3, with 15-16 bp long repeat-variable di-residue (RVD) binding sequences and a 15-bp long spacer. Left and right arm TALEN pair constructs are generated with the help of Golden Gate TALEN assembly strategy as published (T. Cermak, et al. (2015) Methods Mol Biol 1239, 133-159). TALEN mRNAs are synthesized by in vitro message machine kit (Invitrogen). To create indel mutations, 200 pg of each TALEN mRNA is injected into single-cell stage embryos. Injected embryos are grown and screened for germ line transmission. Indel detection at the target locus is performed by High Resolution Melting Curve analysis of the gDNA isolated from caudal fin of adult F1 animals. Indels are analyzed by sequencing of the PCR amplified product using a primer pair binding to the flanking region of the TALEN target. The bns50 allele, harboring a 7-nt deletion, is selected for study.

Quantification of Swim Capacity.

Zebrafish are exercised in groups of 8-12 in a 5 L swim tunnel respirometer device (Loligo). After 20 minutes of acclimation inside the enclosed tunnel, water current velocity is increased every two minutes, and fish swim against the current until they reach exhaustion. Exhausted animals are removed from the chamber without disturbing the remaining fish, while others continue to swim. Swim time and current velocity at exhaustion are recorded. Results are expressed as means±SEM. An unpaired two-tailed Student's t-test with Welch correction is performed using Prism software to determine statistical significance between groups.

Characterization of the ctfga Mutant.

ctgfa mutant (ctgfa^{bns50/bns50}) animals are adult viable, although they survive at a lower rate than their wild-type or heterozygous clutchmates. To assess functional recovery in ctgfa mutants, the capacity (time) of uninjured animals to swim against increasing water currents in an enclosed swim tunnel is evaluated (J. Wang et al. (2011) Development 138, 3421-3430). Adult uninjured ctgfa mutant, heterozygous, and wild-type clutchmates show comparable swim capacities (FIG. 5B), indicating that motor function is not disrupted in ctgfa mutants.

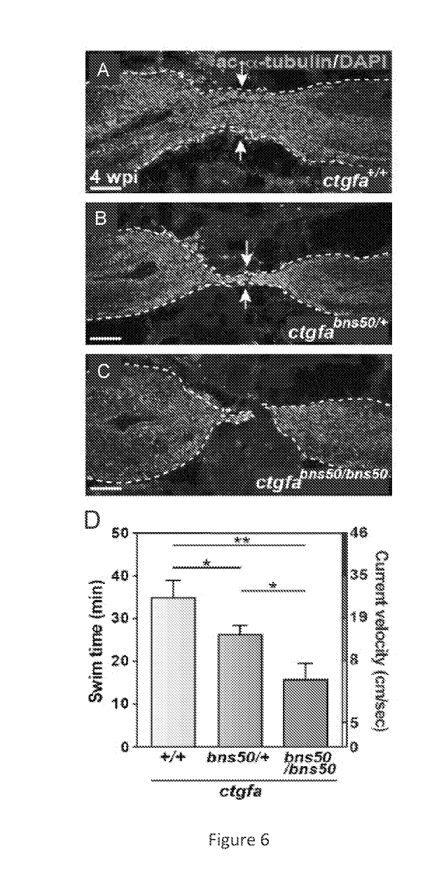

SC transections are performed on ctgfa mutant fish and their morphological and functional recovery assessed by axonal acetyl-α-tubulin staining and swim assays, respectively. Axonal acetyl-α-tubulin staining is a marker for SC repair and regeneration (Goldman D, et al. (2001) Transgenic Res. 10(1):21-33; Diekmann H, et al. (2015) Front Cell Neurosci. 9:118.) Anatomical SC regeneration is markedly impaired at 4 wpi in ctgfa mutant animals compared to heterozygous or wild-type clutchmates (FIG. 6, A-C). Swim capacity is also greatly diminished in ctgfa mutant animals compared to heterozygous and wild-type clutchmates, indicating a lack of functional recovery after SC injury (FIG. 6D).

### Example 4: Enhanced Glial Bridging and Regeneration Following CTGF Overexpression

Generation of Transgenic hsp70:ctgfa Zebrafish.

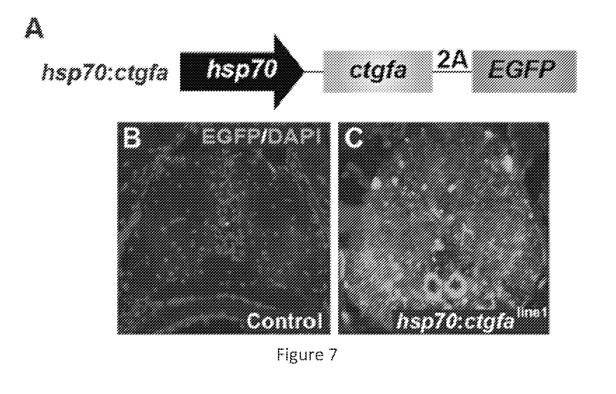

To examine the effects of ctgfa augmentation during SC regeneration, transgenic fish are generated that express full-length ctgfa under control of a heat shock promoter (hsp70:ctgfa) (FIG. 7) (See, e.g., Y. Lee, et al. (2005) Development 132, 5173-5183). The following primers are used to amplify full-length ctgfa from 2 dpf zebrafish cDNA: EcoRV forward primer 5′-ccgatatcgccaccatgttttctggaatgactcaaagtgtgattgc-3′ (SEQ ID NO: 5) and ClaI reverse primer 5′-ccgatatcgccaccatgttttctggaatgactcaaagtgtgattgc-3′ (SEQ ID NO: 6). The amplified fragment is cloned into the hsp70-2A-EGFP vector and then co-injected with I-SceI into one-cell stage wild-type embryos. Multiple founders are isolated, propagated, and screened for germline transition of the transgene. hsp70:ctgfa animals were analyzed as hemizygotes.

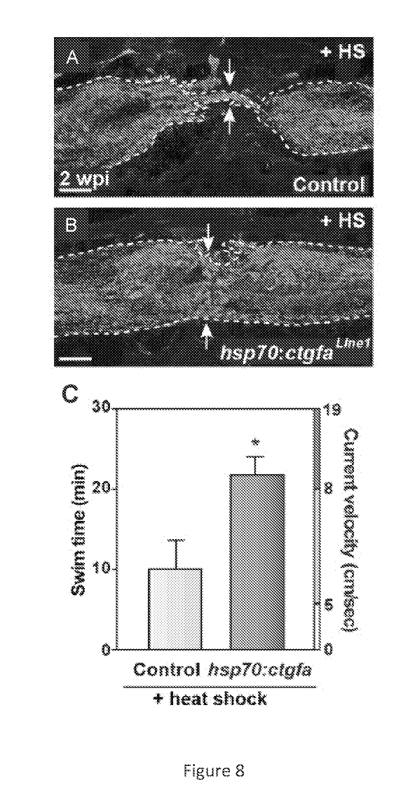

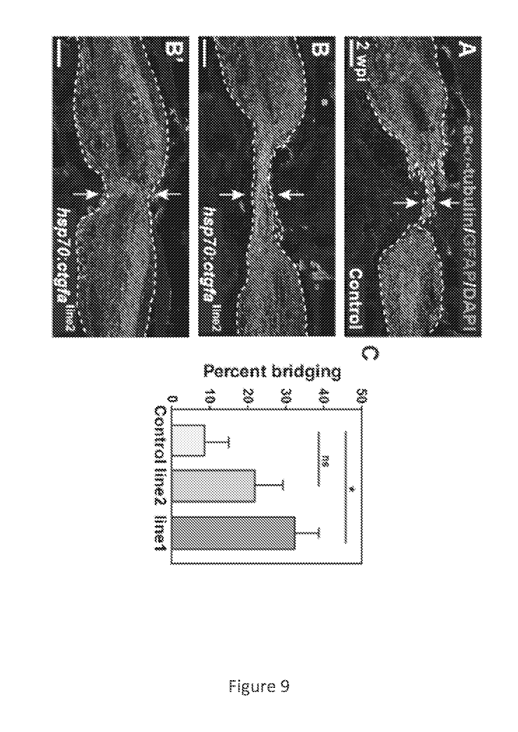

To examine the impact of ctfga overexpression on glial bridging and regeneration, hsp70:ctgfa and control animals are injured, transgene expression induced by daily heat shocks starting at 2 dpi, and animals are examined for SC regeneration at 2 wpi. Histological analysis indicates increased axon regeneration in ctgfa-overexpressing fish compared to transgene-negative controls (FIG. 8A, B and FIG. 9 A-B′). hsp70:ctgfa (line 1) fish, which express a higher level of the transgene relative to a second line of hsp70:ctgfa (line 2) animals, have a greater effect on regeneration (FIG. 9C). Functionally, swim capacity is significantly improved in hsp70:ctgfa animals compared to wild-type clutchmates at 2 wpi (FIG. 8C).

### Example 5: Treatment of Spinal Cord Lesions with Human ctfga Protein Causes SC Regeneration

Evolutionary Conservation of the Ctgf Protein.

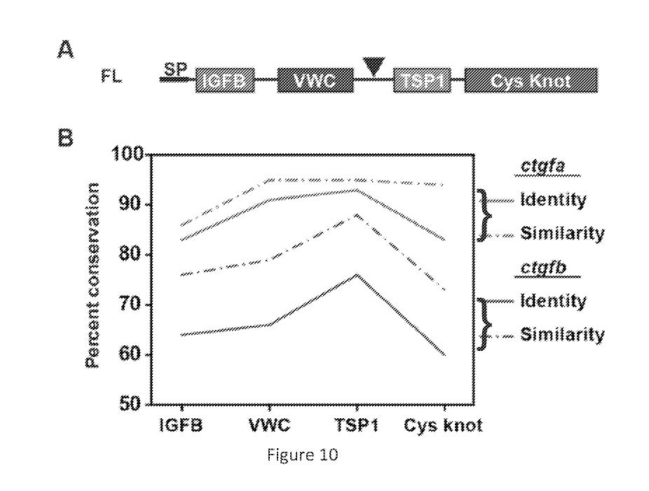

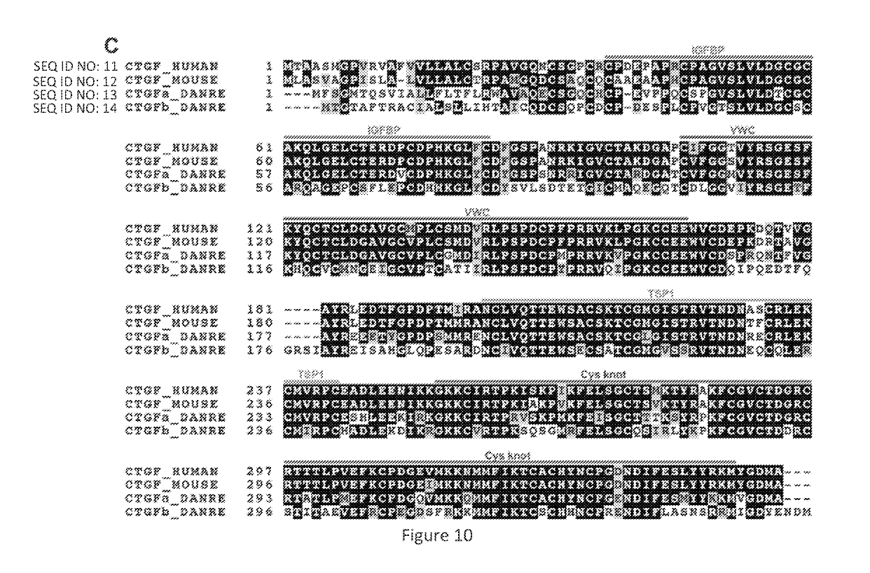

At the amino acid level, human and zebrafish CTGF are 81% identical and 87% similar within the four protein interaction domains (FIG. 10). Therefore, to test the sufficiency of human ctfg in SC regeneration, human recombinant CTGF (HR-CTGF) peptide is applied to injured SCs using a gelfoam sponge and regeneration is assessed functionally (swim test) and structurally.

HR-CTGF Preparation and Gelfoam Application.

A modified version of the spinal cord transection surgery is performed to enable treatment with human recombinant CTGF using gelfoam absorbable gelatin sponges. First, SC transection injury is performed as described above. Briefly, zebrafish 4 months to 1 year old are anaesthetized and placed ventral side down into a moist, slotted sponge. Intraocular scissors are used to make a small incision that penetrates the dorsal side of the skin and resects the spinal cord 4 mm caudal to the brainstem region. Next, gelfoam sponge is prepared and applied onto the injured SC on days 5 and 10 post-injury.

Sterile Gelfoam Absorbable Gelatin Sponge (Pfizer 09-0315-08) is cut into 1 mm³ pieces and soaked for 6 hours at 4° C. with either vehicle (10 □μl of 0.1% BSA in PBS) or recombinant CTGF peptide (10 μl of 100 ng/μl in vehicle, eBioscience, 14-8503-80). The 1 mm³ sponge is then cut into 4-6 smaller pieces. Injured fish at days 5 and 10 post-injury are anaesthetized and placed ventral side down into a moist, slotted sponge. Intraocular scissors are used to expose the injured SC. Vehicle- or CTGF-soaked gelfoam sponges are then applied dorsal to the transected SC. The incision is closed and sealed using Vetbond tissue adhesive material. The incision was closed and sealed using Vetbond tissue adhesive material.

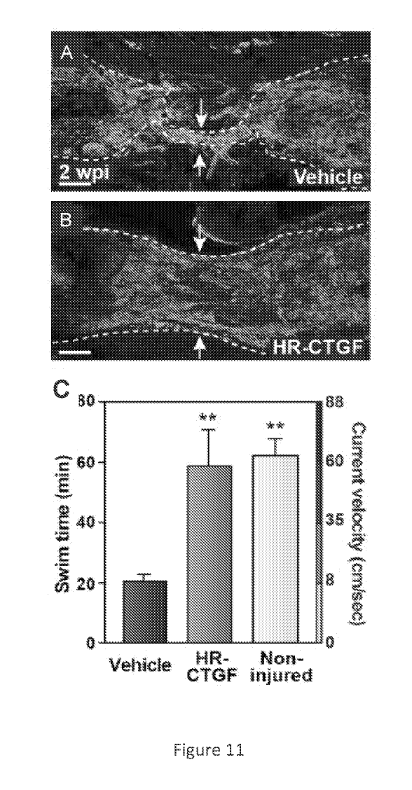

Swim assays are performed at 3 dpi and show that vehicle- and HR-CTGF-treated fish display similar reduced performance, indicating that injuries are comparable across cohorts. In contrast, at 2 wpi, immunohistochemistry reveals enhanced axon regeneration in HR-CTGF-treated animals compared to vehicle controls (FIG. 11A, B). In addition, swim capacity at 2 wpi is conspicuously elevated in HR-CTGF-treated animals, indistinguishable from uninjured animals (FIG. 11C). Thus, human CTGF protein treatment enhances SC regeneration in zebrafish.

### Example 6: CTGF Promotes Glial Bridging Events

To further demonstrate a role for CTGF in SC repair and regeneration, glial bridging is measured by morphometric quantification.

Morphometric Quantification of Glial Bridging.

GFAP immunohistochemistry is performed on serial longitudinal sections. Glial bridge diameter is calculated relative to the diameter of the intact SC at the rostral side of the lesion using ImageJ software. For each fish, percent bridging is calculated for the 3 longitudinal sections that show the most bridging. Percent bridging is then averaged for each cohort. Unpaired two-tailed Student's t-test with Welch correction is performed using the Prism software to determine statistical significance between groups.

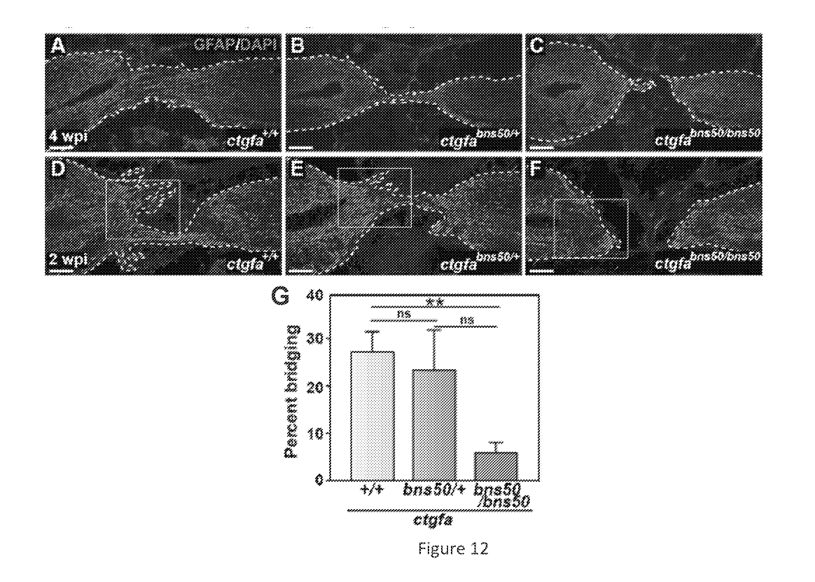

At 2 wpi, wild-type and heterozygous ctgfa animals show signs of glial bridge repair, whereas homozygous ctgfa mutant animals show abrogated bridging (FIG. 12, D-F). This effect is more pronounced at 4 wpi, when wild-type and heterozygous ctgfa animals successfully extend a glial bridge, whereas many homozygous ctgfa mutants fail to bridge their transected SCs (FIG. 12, A-C). By morphometric quantification, ctgfa mutant animals examined at 4 wpi display 74% and 78% less bridging than heterozygous or wild type clutchmates, respectively (FIG. 12G).

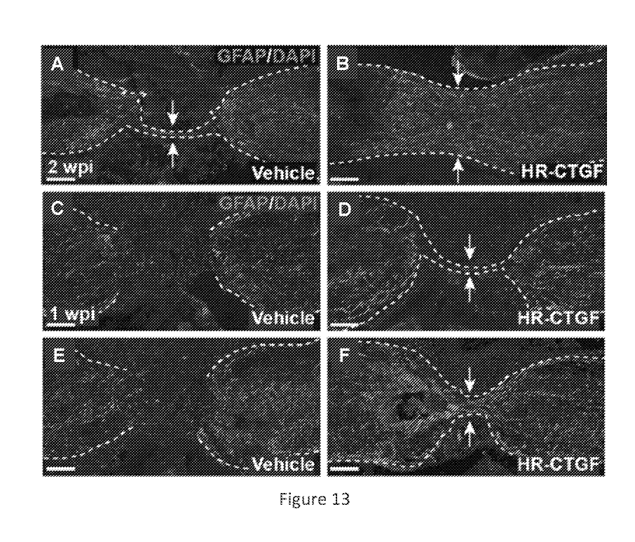

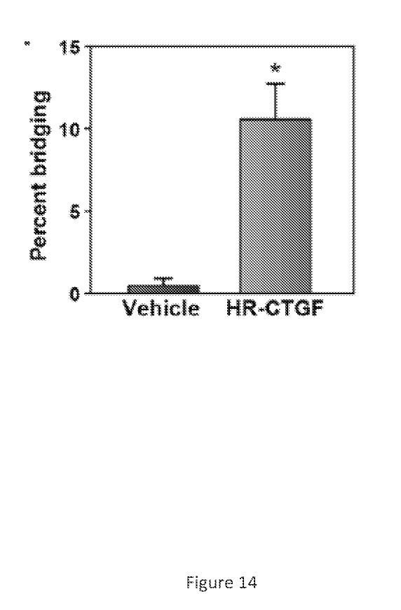

Glial bridging is also examined following HR-CTGF treatment at 1 and 2 wpi (FIG. 13). As early as 1 wpi, increased GFAP expression and enhanced glial bridging in HR-CTGF-treated (FIG. 13 D, F) animals is observed relative to vehicle-treated controls (FIG. 13 C, E). The effect of HR-CTFG is even more pronounced at 2 wpi (compare HR-CTFG treatment in FIG. 13B to vehicle control in FIG. 13A). The results at 1 wpi quantified as a more than 10-fold efficient process after treatment with HR-CTGF (FIG. 14).

### Example 7: CTGF is Induced in Mammalian SC Following Injury and CTGF Overexpression Results in Mammalian Neural Outgrowth

To further demonstrate the role of CTGF in mammalian neural repair and regeneration, the following additional experiments are performed.

Expression of Ctgf after Mouse Spinal Cord Hemisection Injury.

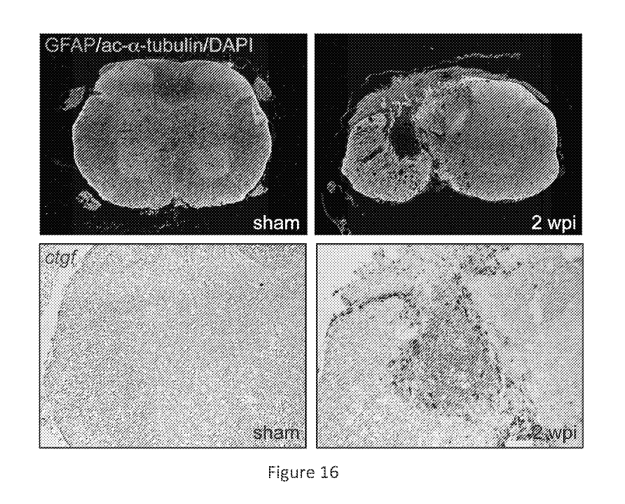

Mouse spinal cord lateral hemisection surgery is performed at the thoracic level (T9) according to methods known in the art. Spinal cord tissue is collected at 2 weeks post-injury for sham and injured mice. RNA in situ hybridization is performed using ctgf transcript to detect ctgf expression after mammalian spinal cord injury. Ctgf expression is induced after hemisection injury at 2 wpi relative to sham treated subject (FIG. 16).

HR-CTGF Treatment and Neurite Outgrowth In Vitro.

Rat retinal ganglion cells (RGCs) are purified by sequential immune-panning from post-natal day 7 rat retinas as previously described by Winzeler A & Wang J T (2013) Cold Spring Harb Protoc 2013(7):643-652. RGCs are cultured at low density in serum-free growth media with or without HR-CTGF for 24 hours, fixed and stained for β-tubulin. RGCs are imaged and analyzed using the neurite outgrowth application of Metamorph software.

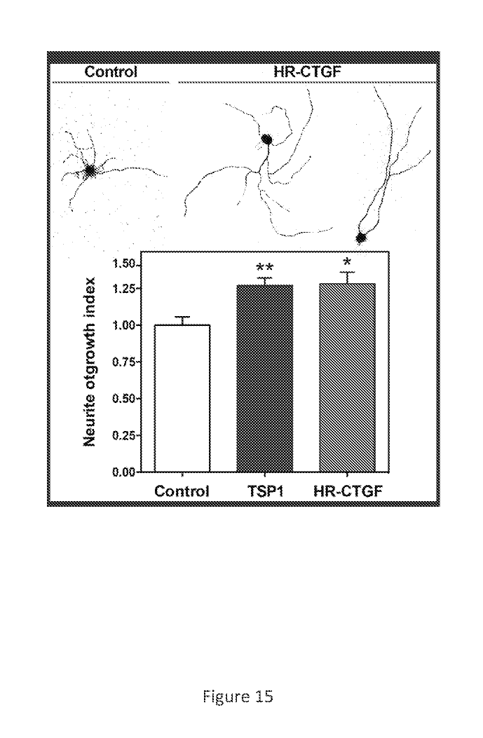

Quantification of neurite outgrowth showed that HR-CTGF significantly increases neurite outgrowth relative to control cultures (FIG. 15). For these experiments, thrombospondin 1, which was previously shown to promote neurite outgrowth, was used as a positive control. These data show that recombinant CTGF treatment induces neurite outgrowth in RGCs in vitro.

One of skill in the art will readily appreciate that the present invention is well adapted to carry out the objects and obtain the ends and advantages mentioned, as well as those inherent therein. The present examples along with the methods, procedures, treatments, molecules, and specific compounds described herein are presently representative of preferred embodiments, are exemplary, and are not intended as limitations on the scope of the invention. Changes therein and other uses will occur to those skilled in the art which are encompassed within the spirit of the invention as defined by the scope of the claims.

1. A pharmaceutical composition comprising a protein or peptide capable of initiating glial bridge formation, regenerating axon tracts along bridging glia regeneration, or generating neuron formation.

2. The pharmaceutical composition of claim 1, wherein the composition comprises connective tissue growth factor (CTGF) peptide.

3. The pharmaceutical composition of claim 2, wherein the peptide comprises human connective tissue growth factor (CTGF) amino acids Lys251-Ala349 (SEQ ID NO: 15).

4. The pharmaceutical composition of claim 1 wherein the protein or peptide is encoded by fn1b, ctgf, mstnb, stc1l, ccl44, or uts2b genes.

5. The pharmaceutical composition of claim 1, further comprising a pharmaceutically acceptable carrier suitable for intravenous, intramuscular, oral, intraperitoneal, intradermal, transdermal, topical, or subcutaneous administration.

6. A method of regenerating spinal cord tissue or ameliorating spinal cord injury in a subject in need thereof, the method comprising administering a therapeutically effective amount of a protein or peptide capable of initiating glial bridge formation, regenerating axon tracts along bridging glia, or generating neuron formation.

7. The method of claim 6, wherein the protein or peptide comprises human connective tissue growth factor (CTFG) protein or peptide.

8. A method of initiating glial bridge formation, stimulating axon tracts along bridging glia regeneration, and/or inducing neuron formation in a tissue of a subject in need thereof by administering CTFG peptide.

9. The method of claim 6 wherein initiating glial bridge formation, stimulating axon tracts along bridging glia regeneration, or inducing neuron formation occurs in spinal cord tissue in the subject.

10. The method of claim 7, wherein the peptide comprises human connective tissue growth factor (CTGF) amino acids Lys251-Ala349 (SEQ ID NO: 15).

11. (canceled)

12. The method of claim 6 wherein the protein or peptide is encoded by fn1b, ctgf, mstnb, stc1l, ccl44, or uts2b genes.

13. The method of claim 6, wherein the subject is a mammal.

14. The method of claim 13, wherein the mammal is a human.

15. A kit useful for the treatment of spinal cord injury in a subject, said kit comprising a therapeutically effective amount of the pharmaceutical composition of claim 1 and instructions for use.

16. The method of claim 6, wherein the subject is a mammal.

17. The method of claim 16, wherein the mammal is a human.

18. The method of claim 8 wherein initiating glial bridge formation, stimulating axon tracts along bridging glia regeneration, or inducing neuron formation occurs in spinal cord tissue in the subject.

19. The method of claim 8, wherein the tissue is spinal cord tissue.

20. The method of claim 8, wherein the CTFG peptide comprises human connective tissue growth factor (CTGF) amino acids Lys251-Ala349 (SEQ ID NO: 15).


SEQUENCE LISTING (15 sequences)

SEQ ID NO: 1 - DNA, 20 bp, Artificial Sequence
  ctgfa forward primer
tgtgattgctctgctgttcc

SEQ ID NO: 2 - DNA, 20 bp, Artificial Sequence
  ctgfa reverse primer
ggtgaggcgattagcttctg

SEQ ID NO: 3 - DNA, 31 bp, Artificial Sequence
  ClaI forward primer
atcgattttggctactttcagctaagactgg

SEQ ID NO: 4 - DNA, 32 bp, Artificial Sequence
  ClaI reverse primer
atcgattctttaaagtttgtagcaaaaagaaa

SEQ ID NO: 5 - DNA, 46 bp, Artificial Sequence
  EcoRV forward primer
ccgatatcgccaccatgttttctggaatgactcaaagtgtgattgc

SEQ ID NO: 6 - DNA, 46 bp, Artificial Sequence; identical to SEQ ID NO: 5
  ClaI reverse primer

SEQ ID NO: 7 - DNA, 57 bp, Danio rerio
ggagagtctttccagagcagttgtaaataccagtcgacgtgtctggacggtgctgta

SEQ ID NO: 8 - protein, 19 aa, Danio rerio
GESFZSSCKYZCTCLDGAV

SEQ ID NO: 9 - DNA, 51 bp, Artificial Sequence
  ctgfa bns50 oligonucleotide
ggagagtctttccagagcagtaccagtgcacgtgtctggacggtgctgtag

SEQ ID NO: 10 - protein, 16 aa, Artificial Sequence
  ctgfa bns50 peptide
GESFQSSTSARVWTVL

SEQ ID NO: 11 - protein, 349 aa, Homo sapiens
MTAASMGPVRVAFVVLLALCSRPAVGQNCSGPCRCPDEPAPRCPAGVSLVLDGCGCCRVCAKQLGELCTERDPCDPHKGLFCDFGSPANRKIGVCTAKDGAPCIFGGTVYRSGESFQSSCKYQCTCLDGAVGCMPLCSMDVRLPSPDCPFPRRVKLPGKCCEEWVCDEPKDQTVVGPALAAYRLEDTFGPDPTMIRANCLVQTTEWSACSKTCGMGISTRVTNDNASCRLEKQSRLCMVRPCEADLEENIKKGKKCIRTPKISKPIKFELSGCTSMKTYRAKFCGVCTDGRCCTPHRTTTLPVEFKCPDGEVMKKNMMFIKTCACHYNCPGDNDIFESLYYRKMYGDMA

SEQ ID NO: 12 - protein, 348 aa, Mus musculus
MLASVAGPISLALVLLALCTRPAMGQDCSAQCQCAAEAAPHCPAGVSLVLDGCGCCRVCAKQLGELCTERDPCDPHKGLFCDFGSPANRKIGVCTAKDGAPCVFGGSVYRSGESFQSSCKYQCTCLDGAVGCVPLCSMDVRLPSPDCPFPRRVKLPGKCCEEWVCDEPKDRTAVGPALAAYRLEDTFGPDPTMMRANCLVQTTEWSACSKTCGMGISTRVTNDNTFCRLEKQSRLCMVRPCEADLEENIKKGKKCIRTPKIAKPVKFELSGCTSVKTYRAKFCGVCTDGRCCTPHRTTTLPVEFKCPDGEIMKKNMMFIKTCACHYNCPGDNDIFESLYYRKMYGDMA

SEQ ID NO: 13 - protein, 345 aa, Danio rerio
MFSGMTQSVIALLFLTFLRWAVAQECSGQCHCPEVPPQCSPGVSLVLDTCGCCRVCAKQLGELCTERDVCDPHKGLYCDYGSPSNRRIGVCTARDGATCVFGGMVYRSGESFQSSCKYQCTCLDGAVGCVPLCGMDIRLPSPDCPMPRRVKVPGKCCEEWVCDSPRQNTFVGSALAAYREEETYGPDPSMMRENCLVQTTEWSACSKTCGLGISTRVTNDNRECRLEKQSRLCMVRPCESHLEEKIRKGKKCIRTPRVSKPMKFEISGCTTTKSYRPKFCGVCTDGRCCTPHRTATLPMEFKCPDGQVMKKQMMFIKTCACHYNCPGENDIFESMYYKKMVGDMA

SEQ ID NO: 14 - protein, 347 aa, Danio rerio
MTGTAFTRACIALSLLIHTAICQDCSQPCDCPDESPLCPVGTSLVLDGCSCCKVCARQAGEPCSFLEPCDHHKGLYCDYSVLSDTETGICMAQEGQTCDLGGVIYRSGETFQPSCKHQCVSMNGEIGCVPTCATIIRLPSPDCPYPRRVQIPGKCCEEWVCDQIPQEDTFQSAVAAYREISAHGLQPESARDNCIVQTTEWSECSATCGMGVSSRVTNDNEQCQLERQTRMCMIRPCHADLEKDIKRGKKCVRTPKSQSGMRFELSGCQSIRLYKPKFCGVCTDDRCCTPQSTITAEVEFRCPEGDSFRKKMMFIKTCSCHHNCPRENDIFLASNSRRMIGDYENDM

SEQ ID NO: 15 - protein, 99 aa, Homo sapiens
KKGKKCIRTPKISKPIKFELSGCTSMKTYRAKFCGVCTDGRCCTPHRTTTLPVEFKCPDGEVMKKNMMFIKTCACHYNCPGDNDIFESLYYRKMYGDMA